(19) United States
(12) Patent Application Publication
(10) Pub. No.: US 2026/0102181 A1
(43) Pub. Date: Apr. 16, 2026

(54) ASPIRATION CATHETER WITH AUTOMATIC SENSING

(71) Applicant: BOSTON SCIENTIFIC SCIMED, INC., Maple Grove, MN (US)

(72) Inventors: Tatum Rubald, Orlando, FL (US); Michael William Nagel, Andover, MN (US); Alyssa Madej, Minnetonka, MN (US); Robert D. Cooper, Andover, MN (US)

(73) Assignee: BOSTON SCIENTIFIC SCIMED, INC., Maple Grove, MN (US)

(21) Appl. No.: 19/357,679

(22) Filed: Oct. 14, 2025

Related U.S. Application Data
(60) Provisional application No. 63/707,403, filed on Oct. 15, 2024.

(51) Int. Cl. A61B 17/3203; A61B 17/00; A61B 17/22; A61B 90/00

(52) U.S. Cl. CPC A61B 17/32037; A61B 2017/00022; A61B 2017/22079; A61B 2090/064

(57) ABSTRACT

Thrombectomy catheters and systems for detecting the present of a clot or thrombus. An illustrative thrombectomy catheter may comprise an effluent return tube comprising a catheter body extending from a proximal end region to a distal end region and including a catheter lumen extending between the proximal end region and the distal end region, a high-pressure fluid supply tube extending through the catheter lumen from the catheter body proximal end region toward the catheter body distal end region, the high-pressure fluid supply tube configured for communication with a fluid source near the catheter body proximal end region, a transition fixture coupled to the proximal end region of the catheter body, the transition fixture including at least one port; and a sensor coupled relative to the catheter body and configured to monitor a flow of fluid through the catheter lumen.

### CROSS-REFERENCE TO RELATED APPLICATION

This application claims the benefit of U.S. Patent Provisional application serial no. 63/707,403, filed October 15, 2024, entitled "ASPIRATION CATHETER WITH AUTOMATIC SENSING”, which is incorporated by reference herein in its entirety.

### TECHNICAL FIELD

The disclosure is directed to aspiration systems. More particularly, the disclosure is directed to an aspiration catheter system for improved clot removal. In some instances, the aspiration catheter may include automatic clot sensing and removal.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices.

In an example, a thrombectomy catheter may include an effluent return tube comprising a catheter body extending from a proximal end region to a distal end region and including a catheter lumen extending between the proximal end region and the distal end region. The catheter may also include a high-pressure fluid supply tube extending through the catheter lumen from the catheter body proximal end region toward the catheter body distal end region, with the high-pressure fluid supply tube configured for communication with a fluid source near the catheter body proximal end region. Additionally, the catheter may have a transition fixture coupled to the proximal end region of the catheter body, with the transition fixture including at least one port. Furthermore, the catheter may include a sensor coupled relative to the catheter body and configured to monitor a flow of fluid through the catheter lumen.

Alternatively or additionally to any of the examples above, in another example, the sensor may be a pressure sensor.

Alternatively or additionally to any of the examples above, in another example, the pressure sensor may be secured to the at least one port.

Alternatively or additionally to any of the examples above, in another example, the pressure sensor may be releasably coupled to the at least one port.

Alternatively or additionally to any of the examples above, in another example, the pressure sensor may be configured to measure a pressure of a fluid in the catheter lumen.

Alternatively or additionally to any of the examples above, in another example, the sensor may be a non-contact flow sensor.

Alternatively or additionally to any of the examples above, in another example, the non-contact flow sensor may be in contact with an outer surface of the catheter body.

Alternatively or additionally to any of the examples above, in another example, the non-contact flow sensor may be releasably secured to the outer surface of the catheter body.

Alternatively or additionally to any of the examples above, in another example, the non-contact flow sensor may comprise a load cell.

Alternatively or additionally to any of the examples above, in another example, the load cell may be disposed within a housing.

Alternatively or additionally to any of the examples above, in another example, a loading platform of the load cell may be in contact with the outer surface of the catheter body.

Alternatively or additionally to any of the examples above, in another example, the non-contact flow sensor may comprise an ultrasound doppler flow meter.

Alternatively or additionally to any of the examples above, in another example, the non-contact flow sensor may be configured to measure a flow rate of a fluid in the catheter lumen.

Alternatively or additionally to any of the examples above, in another example, the thrombectomy catheter may further include a data acquisition device electronically coupled to the sensor.

In another example, a thrombectomy system may include a processor coupled to a pump and a thrombectomy catheter. The thrombectomy catheter may include a catheter body extending from a proximal end region to a distal end region and including a catheter lumen extending between the proximal end region and the distal end region. The catheter may also include a high-pressure fluid supply tube extending through the catheter lumen from the catheter body proximal end region toward the catheter body distal end region, with the high-pressure fluid supply tube configured for communication with a fluid source near the catheter body proximal end region. Additionally, the catheter may include a sensor releasably coupled relative to the catheter body and configured to monitor a flow of fluid through the catheter lumen.

Alternatively or additionally to any of the examples above, in another example, the processor may be operably coupled to the sensor.

In another example, a method for detecting a thrombus may include inserting a thrombectomy catheter into a lumen. The thrombectomy catheter may include a catheter body extending from a proximal end region to a distal end region and including a catheter lumen extending between the proximal end region and the distal end region. The catheter may also include a high-pressure fluid supply tube extending through the catheter lumen from the catheter body proximal end region toward the catheter body distal end region, with the high-pressure fluid supply tube configured for communication with a fluid source near the catheter body proximal end region. Additionally, the catheter may include a sensor releasably coupled relative to the catheter body and configured to monitor a flow of fluid through the catheter lumen. The method may further include determining a pressure or a flow rate of the fluid in the catheter lumen from a signal from the sensor, and determining the distal end region of the catheter body is in contact with a thrombus when the pressure or flow rate is less than a predetermined threshold for a first predetermined length of time.

Alternatively or additionally to any of the examples above, in another example, the method may include determining the thrombectomy catheter is actively removing the thrombus when the pressure or flow rate is less than the predetermined threshold for a second predetermined length of time greater than the first predetermined length of time.

Alternatively or additionally to any of the examples above, in another example, the sensor may be a pressure sensor.

Alternatively or additionally to any of the examples above, in another example, the sensor may be a non-contact flow sensor.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an illustrative thrombectomy system;

FIG. 2 is a partially exploded perspective view of a portion of the thrombectomy system of FIG. 1;

FIG. 3 is a longitudinal cross-sectional view of a distal end region of the illustrative thrombectomy catheter of FIG. 1;

FIG. 4 is a side view of a portion of the thrombectomy system shown in FIG. 1;

FIG. 5 is an illustrative graph of illustrative pressure data obtained at the pressure sensor of the thrombectomy catheter of FIG. 3 over time during a thrombectomy procedure;

FIG. 6 is a perspective view of an illustrative non-contact flow sensor positioned around an outer surface of an effluent return tube; and

FIG. 7 is a side view of another illustrative non-contact flow sensor in contact with an outer surface of an effluent return tube.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the shaft of the catheter. However, prolonged operation of a thrombectomy system may result in the detrimental removal of excess blood from the veins or arteries of a patient. Accordingly, it may be desirable to design a thrombectomy system which operates (e.g., activates to remove thrombus, plaque, blood, etc.) only when the distal tip of the system senses the presence of clot material. Thrombectomy systems which operate (e.g., activate to remove thrombus, plaque, blood, etc.) only when the distal tip of the system senses the presence of clot material are disclosed herein.

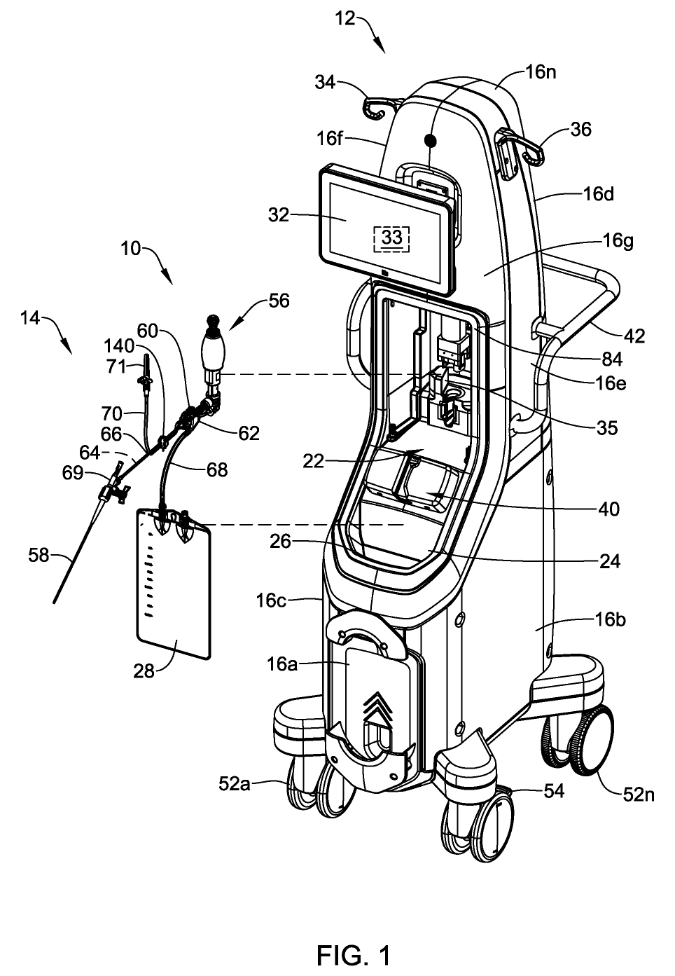

FIG. 1 is a perspective view of an illustrative thrombectomy system 10. The thrombectomy system 10 may include a control console 12, including a drive unit, and a pump/catheter assembly 14. In some instances, the pump/catheter assembly 14 may be a disposable single use device in which a new pump/catheter assembly 14 may be used with the console 12 for each medical procedure. The console 12 may include a housing enclosing the internal structure of the console 12. Shown on the console 12 are a plurality of removable panels 16a-16n about and along the console 12 enclosing the internal structure of the console 12. An illustrative console 12 is described in commonly assigned U.S. Patent Number 7,935,077, titled THROMBECTOMY CATHETER DEPLOYMENT SYSTEM, the disclosure of which is hereby incorporated by reference. Centrally located in the console 12 and aligned to the lower region of the panel 16g may be automatically opening loading bay door assemblies (not explicitly shown) which open to expose the interior of the console 12 to provide access to a carriage assembly 22 and close during use of the pump/catheter assembly 14. Illustrative loading bay doors and assemblies are described in commonly assigned U.S. Patent Application Number 18/604,956, titled THROMBECTOMY SYSTEM WITH LOADING BAY DOORS, the disclosure of which is hereby incorporated by reference.

The console 12 may include a catch basin or drip tray 24 for collecting fluid leakage from the components of the pump/catheter assembly 14. In some instances, the drip tray 24 may be removable. Other configurations of catch basins are also contemplated. The drip tray 24 and/or a receptacle 26 may collectively support and accommodate an effluent collection bag, such as effluent collection bag 28 of the pump/catheter assembly 14. In other instances, the console 12 may include a different structure, such as a hook for hanging the effluent collection bag 28 from, or a shelf for setting the effluent collection bag 28 on. An effluent waste tube 68 may also be positioned in an outflow or a roller pump 40 between the tube guides with the effluent collection bag 28 connected to the effluent waste tube 68. The effluent collection bag 28 may be suitably positioned for collecting effluent during the medical procedure. Pump rollers (not shown) of the roller pump 40 may rotatably engage the effluent waste tube 68 to control effluent fluid flow through the effluent waste tube 68 to the effluent collection bag 28.

In instances where the carriage assembly 22 is movable, a carriage assembly activation switch (not explicitly shown) may be provided with the console 12, such as located on a panel 16g, to selectively position the carriage assembly 22 inwardly or outwardly. In other instances, the carriage assembly 22 may be positioned or moved using a control panel and/or user interface 32. A user interface 32, including memory and/or processing capabilities, may be provided with the console 12, such as located at the upper region of the console 12 between the upper regions of the upper side panels 16e and 16f. The user interface 32 may be a guided user interface (GUI) including a touch screen display to allow a user to provide input to the user interface 32 and view information on a same display screen. However, this is not required. In other instances, the user input may be separate from the display screen.

Saline bag hooks 34 and 36 may extend through the panels 16e and 16f to hang saline bags therefrom. The console 12 may include a handle 42 as well as a plurality of wheels 52a-52n and brake pedals 54 for wheel lockage to assist in maneuvering the console 12 by medical personnel.

In FIG. 1, the pump/catheter assembly 14 is shown detached from the console 12. The pump/catheter assembly 14 includes an inflow pump 56 and a thrombectomy device or catheter 58. In some embodiments, the inflow pump 56 may be configured to provide fluid inflow through the thrombectomy device 58. During use, a portion of the pump/catheter assembly 14 may be secured within a portion of the console 12. In some embodiments, the pump/catheter assembly 14 may include a bubble trap 60 attached to the inflow pump 56, a connection manifold assembly 62 connected to the bubble trap 60, a fixture 140, an effluent return tube 66 connected between the connection manifold assembly 62 and the thrombectomy device 58, a high-pressure fluid supply tube 64 attached between the output of the inflow pump 56 and the thrombectomy device 58 which may be arranged inside the effluent return tube 66, a transition fixture 69 between the distal end of the effluent return tube 66 and the proximal end of the thrombectomy device 58, an effluent waste tube 68 connecting the effluent collection bag 28 to the connection manifold assembly 62, and a fluid supply tube 70 having a bag spike 71 connecting a fluid supply bag (e.g., a saline bag) (not explicitly shown) to the connection manifold assembly 62. The fluid supply tube 70 may be in fluid communication with the interior of the bubble trap 60 to provide fluid from the fluid supply bag to the inflow pump 56 and then to the thrombectomy device 58 through the high-pressure fluid supply tube 64.

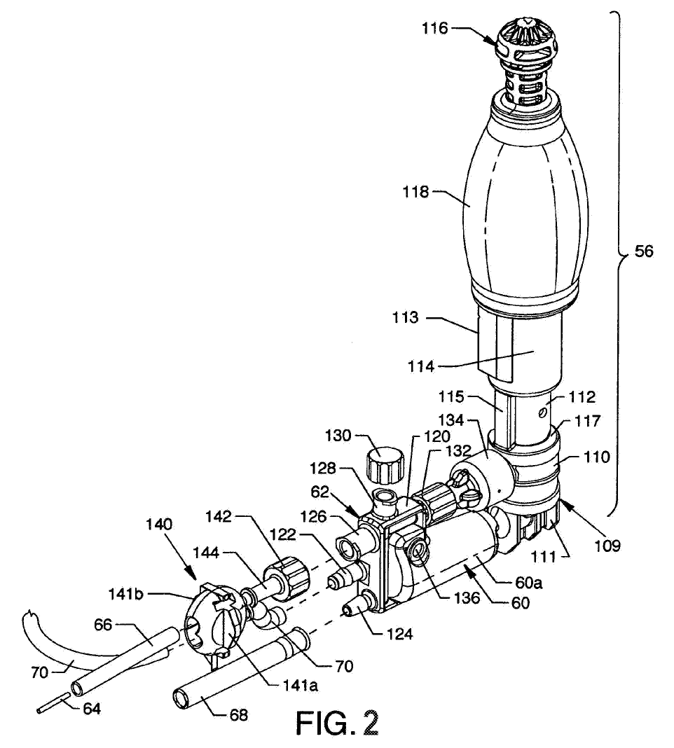

The console 12 may include a reciprocating linear actuator 84 configured to engage a pump piston head 116 (see, for example, FIG. 2) of the inflow pump 56 when the inflow pump 56 is engaged with the carriage assembly 22. The reciprocating linear actuator 84 may be disposed within the interior of the drive unit 12 and may be aligned with the pump piston head 116 (e.g., FIG. 2) when the inflow pump 56 is disposed within the interior of the drive unit 12. The reciprocating linear actuator 84 may be actuated such that reciprocating (e.g., up and down) strokes of the reciprocating linear actuator 84 drive the inflow pump 56 in response to activation of a user activation switch (not explicitly shown) or a control command from a controller 33. In some embodiments, the user activation switch may be a foot switch. In some embodiments, when the user activation switch is depressed, the reciprocating linear actuator 84 and/or the inflow pump 56 is activated and/or operates, and when the user activation switch is released, the reciprocating linear actuator 84 and/or the inflow pump 56 is stopped and/or ceases operation.

The console 12 may include a controller 33 in electronic communication with the user interface 32, the reciprocating linear actuator 84, the inflow pump 56, the roller pump 40, and/or the thrombectomy device 58. In some cases, the controller 33 may be a part of or otherwise incorporated into the user interface 32. In some embodiments, the console 12 and/or the controller 33 may include a data acquisition device 35. In some embodiments, the data acquisition device 35 may be disposed within the interior of the drive unit 12. For example, the data acquisition device 35 may be positioned on or near the carriage assembly 22. In some embodiments, the data acquisition device 35 may be configured for wireless communication. Other configurations are also contemplated. The data acquisition device 35 may be a radiofrequency identification reader and/or a barcode reader. Other types of data acquisition devices 35 may be used, as desired. In some embodiments, the user activation switch may be in electronic communication with the drive unit 12 and/or the controller 33. In some embodiments, the user activation switch may be in electronic communication with the drive unit 12 and/or the controller 33 via a wire or cable. In some embodiments, the user activation switch may be in electronic communication with the drive unit 12 and/or the controller 33 wirelessly.

FIG. 2 is a partially exploded perspective view of several components of the pump/catheter assembly 14 (e.g., FIG. 1) generally including the inflow pump 56, the bubble trap 60, the connection manifold assembly 62, and the fixture 140. The inflow pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 (e.g., FIG. 1) to retain the inflow pump 56 within and/or in engagement with the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example.

In some embodiments, a data plate 113 may also be included on the pump/catheter assembly 14, such as on the top body 114 for example, for the inclusion of a barcode, a radiofrequency identification (RFID) tag, a data storage chip, informational displays, etc. to store, communicate, and/or otherwise determine specifications and/or operational parameters associated with the pump/catheter assembly 14, the thrombectomy device 58, the inflow pump 56, etc. and/or components thereof. In at least some embodiments, the data acquisition device 35 (e.g., FIG. 1) may be configured to communicate with the data plate 113 (e.g., the barcode, the RFID tag, the data storage chip, etc.) to obtain specifications, operational parameters, and/or identifying information associated with the pump/catheter assembly 14, the thrombectomy device 58, the inflow pump 56, etc., and/or components thereof.

In some embodiments, the inflow pump 56 may include a hemispherically-shaped pump piston head 116 having a flexible boot 118 connected to and extending between the top body 114 and the pump piston head 116. In some instances, the lower portion 111 of the base 109 may serve as a mount for a first end of the bubble trap 60.

The connection manifold assembly 62 may be secured directly to a second end of the bubble trap 60 and in some instances may include a bracket 120 to which is attached a vertically oriented tubular manifold having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connectingly extending between the connection manifold assembly 62 and the upper portion 110 of the base 109.

The bubble trap 60 may include two mating halves of which a first bubble trap half 60a is shown. A hydrophobic filter 136 may be included at the upper forward region of the first bubble trap half 60a. In some embodiments, a second hydrophobic filter may be included on the second bubble trap half (not explicitly shown) which opposes the hydrophobic filter 136 on the first bubble trap half 60a.

The fixture 140, and components associated therewith, assists in support and connection of the effluent return tube 66 to the effluent return port 126 by a connector 142 combined continuously with a connection tube 144, and also assists in support, passage, and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting the roller pump 40 (e.g., FIG. 1) provided within the drive unit 12, such as located in the carriage assembly 22 or adjacent thereto.

In some embodiments, the effluent waste tube 68 may be positioned within and/or through the outflow pump with the effluent collection bag 28 (e.g., FIG. 1) connected to the effluent waste tube 68. The effluent collection bag 28 may be suitably positioned for collecting effluent during the procedure. In some embodiments, pump rollers of the roller pump 40 may engage the effluent waste tube 68 to control effluent fluid flow through the effluent waste tube 68 from the effluent outlet port 124 to the effluent collection bag 28. In other embodiments, the pump rollers of the roller pump 40 may be disengaged from the effluent waste tube 68 to allow for free flow of the effluent. In some embodiments, other pump types and/or configurations may be used in place of the roller pump.

At an appropriate time, the thrombectomy device 58 may be subjected to a priming procedure to purge the thrombectomy device 58 of any air. For example, the tip of the thrombectomy device 58 may be placed in a bowl of sterile saline, or other fluid, and the inflow pump 56 may be operated by action of the reciprocating linear actuator 84 (such as by activating and/or depressing the user activation switch) to prime the thrombectomy device 58. Thereafter, medical personnel may insert the thrombectomy device 58 into the vasculature of the patient, and operation of the thrombectomy system 10 incorporating the user interface 32 and the user activation switch can begin, as desired. The reciprocating linear actuator 84 is actuated according to the operating parameters to influence fluid inflow pressures, pump speed, flow rates, and the like to operate the inflow pump 56 to deliver pressurized fluid to the thrombectomy device 58 via the high-pressure fluid supply tube 64 residing in the effluent return tube 66. Supply fluid is routed through the bubble trap 60, may be pressurized by the inflow pump 56, and is routed through the high-pressure fluid supply tube 64 to the thrombectomy device 58 for use in a thrombectomy or other related procedure. Effluent may be returned through the effluent return tube 66 to the connection manifold assembly 62 for collection in the effluent collection bag 28 (e.g., FIG. 1) through the effluent waste tube 68.

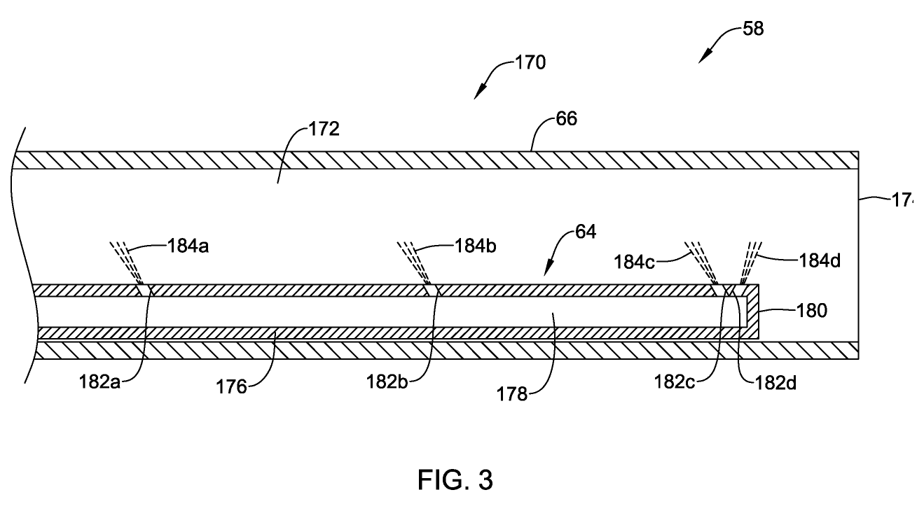

FIG. 3 is a cross-sectional view of a distal end region 170 of the illustrative thrombectomy catheter 58. The thrombectomy catheter 58 may include the effluent return tube 66. The effluent return tube 66 may be a tubular member or catheter body extending from a proximal end region (not explicitly shown) configured to remain outside the body to a distal end region 170. In other embodiments, the thrombectomy catheter 58 may include a separate tubular member or catheter body that is fluidly coupled with the effluent return tube 66. A lumen 172 may extend from the proximal end region to the distal end region 170 of the effluent return tube 66. The effluent return tube 66 may terminate at a distally facing distal opening 174 at the distal end of the effluent return tube 66. In some instances, the distal opening 174 may be in a plane that extends generally orthogonal to a longitudinal axis of the effluent return tube 66. In other instances, the distal opening 174 may be in a plane that extends generally oblique to a longitudinal axis of the effluent return tube 66. Generally, the distal opening 174 may be an entrainment inflow orifice. While not explicitly shown, the effluent return tube 66 may include one or more markers (e.g., radiopaque marker bands) disposed along the effluent return tube 66. Further, while not explicitly shown, in some embodiments, the effluent return tube 66 may include one or more openings extending through a sidewall thereof, if desired.

The thrombectomy catheter 58 may further include the high-pressure fluid supply tube 64. In other embodiments, the thrombectomy catheter 58 may include a separate tubular member or catheter body that is fluidly coupled with the high-pressure fluid supply tube 64. The high-pressure fluid supply tube 64 may be disposed within and extend through the lumen 172 of the effluent return tube 66. The high-pressure fluid supply tube 64 may include a supply tube wall 176 defining a lumen or fluid pathway 178 extending therethrough. In at least some instances, the high-pressure fluid supply tube 64 may have a closed distal end 180. Because of this, fluid may be able to pass distally through the fluid pathway 178 but does not exit the distal end. The high-pressure fluid supply tube 64 may extend along a length of the effluent return tube 66 with the distal end 180 located within the lumen 172 of the effluent return tube 66 proximal to the distal opening 174 at the distal end of the effluent return tube 66. A proximal end of the high-pressure fluid supply tube 64 may be in fluid communication with the pump 56 described herein, to provide high-pressure fluid to the fluid pathway 178 of the high-pressure fluid supply tube 64.

A plurality of jet orifices 182a-d (collectively, 182) may be defined along the supply tube wall 176. For example, the supply tube wall 176 may include two, three, four, five, six, or more jet orifices 182. The jet orifices 182 may be spaced along the supply tube wall 176 at any desired intervals. For example, each of the jet orifices 182 may be equidistantly spaced from adjacent jet orifices 182 along the length of the supply tube wall 176. In other instances, the jet orifices 182 may be arranged such that the spacing between adjacent jet orifices 182 near the distal end of the supply tube wall 176 is closer than the spacing between adjacent jet orifices 182 near the proximal end of the supply tube wall 176. An illustrative console high-pressure supply tube having jet orifices is described in commonly assigned U.S. Patent Publication Number 2024/0245423, titled ASPIRATION CATHETER WITH DISTALLY DIRECTED FLUID JET, the disclosure of which is hereby incorporated by reference.

Infusion of motive fluid through the lumen 178 of the supply tube wall 176 may result in fluid being jetted through the jet orifices 182 and the generation of a proximally directed aspiration force. At least some of the jet orifices 182a-c may be angled in a proximal direction or otherwise designed to infuse fluid (e.g., a motive fluid, a liquid, a gas or air, steam, a fluid with particles disposed therein, or the like) through the jet orifices 182a-c and into the lumen 172 of the effluent return tube 66 in a generally proximal direction as depicted by lines 184a-c representing motive jetted fluid projecting generally proximally from the jet orifices 182a-c. For example, each of the jet orifices 182a-c may be arranged at an acute angle to the longitudinal axis of the supply tube wall 176 such that the jet orifices 182a-c angle in a proximal direction. In some embodiments, one or more of the jet orifices 182d may be designed to infuse fluid (e.g., a motive fluid, a liquid, a gas or air, steam, a fluid with particles disposed therein, or the like) through the jet orifice(s) 182d and into the lumen 172 of the effluent return tube 66 in a generally distal direction as depicted by lines 184d representing motive jetted fluid projecting generally distally from the jet orifice 182d. For example, the jet orifice 182d may be arranged at an oblique angle to the longitudinal axis of the supply tube wall 176 such that the jet orifice 182d angles in a distal direction. It is contemplated that an angle of the jet orifices 182 and thus the motive jetted fluid 184 may be varied to adjust the velocity of the fluid exiting the jet orifices 182. The supply tube wall 176 may include one or more, or a plurality of proximally oriented or directed jet orifices 182a, 182b, 182c (i.e., jet orifices configured to direct fluid infused through the lumen 178 of the supply tube wall 176 in a proximal direction) and the supply tube wall 176 may include one or more, or a plurality of distally oriented or directed jet orifices 182d (i.e., jet orifices configured to direct fluid infused through the lumen 178 of the supply tube wall 176 in a distal direction). The distally projecting jet orifice 182d may be the distalmost jet orifice, with the proximally projecting jet orifices 182a-c positioned proximal of the distally projecting jet orifice 182d. However, this is not required. In some embodiments, the distally projecting jet orifice 182d may be positioned proximal to at least one proximally projecting jet orifice 182a-c. The distally projecting jet orifice(s) 182d may break up particles as they are drawn into the lumen 172 of the effluent return tube 66 while the proximally projecting jet orifices 182a-c may move particles proximally along the effluent return tube 66.

The performance of the thrombectomy catheter 58 and the high-pressure fluid supply tube 64 may be directly related to the velocity of the motive jetted fluid 184a-d exiting the jet orifices 182 and the shear-induced turbulent flux created by the jetted motive fluid 184a-d. For example, the more powerful the jetted motive fluid 184a-d, the higher the aspiration rates may be. It is further contemplated that the performance of the jet-powered aspiration catheter 58 may be directly related to the speed at which the clot can be entrained into the catheter 58, macerated, and removed from the body. Any clogging that occurs within the effluent return tube 66 may reduce or completely stop the removal of the clot. The addition of the distally projecting jet orifice 182d may macerate or liquify any clot that enters the distal opening 174 of the effluent return tube 66 thus helping prevent clogging. For example, at the point of impingement of the distally oriented motive jetted fluid 184d the motive jetted fluid 184d may deflect distally creating flow out the tip of the distal opening 174 of the effluent return tube 66, effectively macerating any clot that enters the tip of the device, and eliminating or reducing risk of the distal opening 174 of the effluent return tube 66 becoming blocked or clogged. It is contemplated that the properties (size, shape, angle, number, spacing, etc.) of the jet orifices 182 may be varied to obtain a fluid velocity that creates an optimum de-clogging effect without hindering the proximal flow of a clot within the lumen 172 of the effluent return tube 66 or the clot evacuation rate.

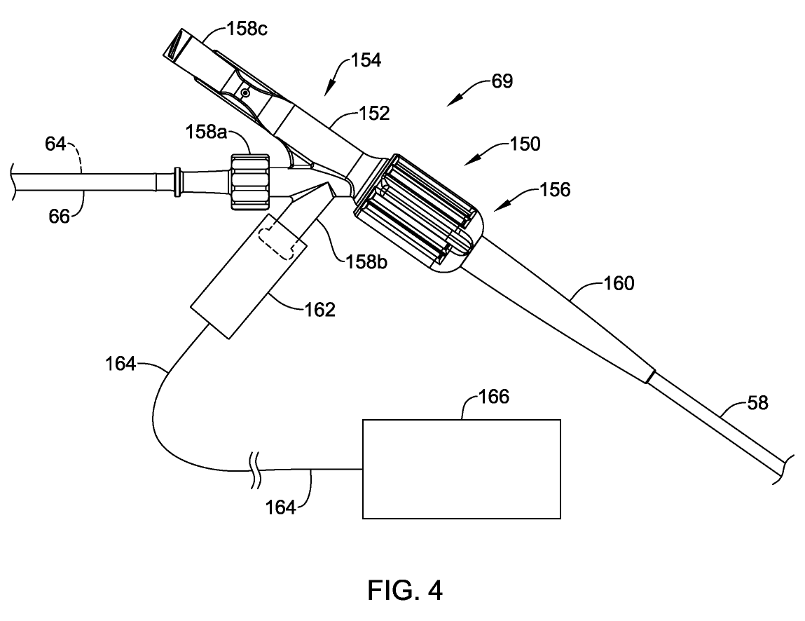

FIG. 4 is a side view of the illustrative transition fixture 69 of the thrombectomy catheter 58. The transition fixture 69 may include a manifold 150 including a body portion 152. The manifold 150 may extend from a proximal end region 154 to a distal end region 156. The proximal end region 154 may include one or more ports 158a, 158b, 158c for fluidly coupling the thrombectomy catheter 58 with other devices or portions of the system. For example, a first port 158a may be fluidly coupled to the effluent return tube 66 with the high-pressure fluid supply tube 64 extending therethrough. A second port 158b may be a contrast injection port. These are just some examples. The ports 158a-c may be arranged in any configuration and/or configured to be coupled to various portions of the system 14. Further, the manifold 150 may include fewer than three or more than three ports 158a-c, as desired. The distal end region 156 of the manifold 150 may be coupled to a strain relief 160 which in turn is coupled to the thrombectomy catheter 58. One or more of the ports 158a-c may be in fluid communication with the lumen 172 of the effluent return tube 66 and/or the lumen 178 of high-pressure fluid supply tube 64. For example, contrast injected through the second port 158b may enter the lumen 172 of the effluent return tube 66 and/or the lumen 178 of high-pressure fluid supply tube 64.

The transition fixture 69 may include a pressure sensor 162 releasably coupled with the second port 158b and configured to monitor a flow of fluid through the lumen 172 of the effluent supply tube 66. However, the pressure sensor 162 may be releasably coupled with other ports 158a, 158c, as desired. The pressure sensor 162 may be in fluid communication with the lumen 172 of the effluent return tube 66. In one illustrative example, the pressure sensor 162 may include a diaphragm with strain gauges arranged in a Wheatstone bridge configuration. An illustrative pressure sensor 162 may be the Model S-20 commercially available from WIKA USA, Lawrenceville, GA. When pressure (e.g., pressure in the lumen 172 of the effluent return tube 66) is applied to the diaphragm, the diaphragm deforms causing the strain gauges to change their resistance. This change in resistance unbalances the Wheatstone bridge, generating a small differential voltage. This voltage is then amplified by an instrumentation amplifier to produce a higher-level signal. The amplified signal is processed and converted into a standardized output signal, such as 4-20 milliamps (mA) or 0-10 volts (V). The voltage signal can then be converted to pressure by using a data acquisition device (DAQ) 166. For example, the voltage signal is proportional to the pressure in the lumen 172. The pressure sensor 162 may be electrically and communicatively coupled to the DAQ 166 with an electrical connection 164. In some embodiments, the DAQ 166 may be a part of or positioned within the control console 12. The DAQ 166 may a separate component from the controller 33. Alternatively, the DAQ may be incorporated into the controller 33. In yet another embodiment, the DAQ 166 may be a separate structure that is electrically and communicatively coupled to the controller 33 via a wired or wireless connection. It is contemplated that the pressure sensor 162 and/or the DAQ 166 may be supplied with the pump/catheter assembly 14 or may be separately supplied.

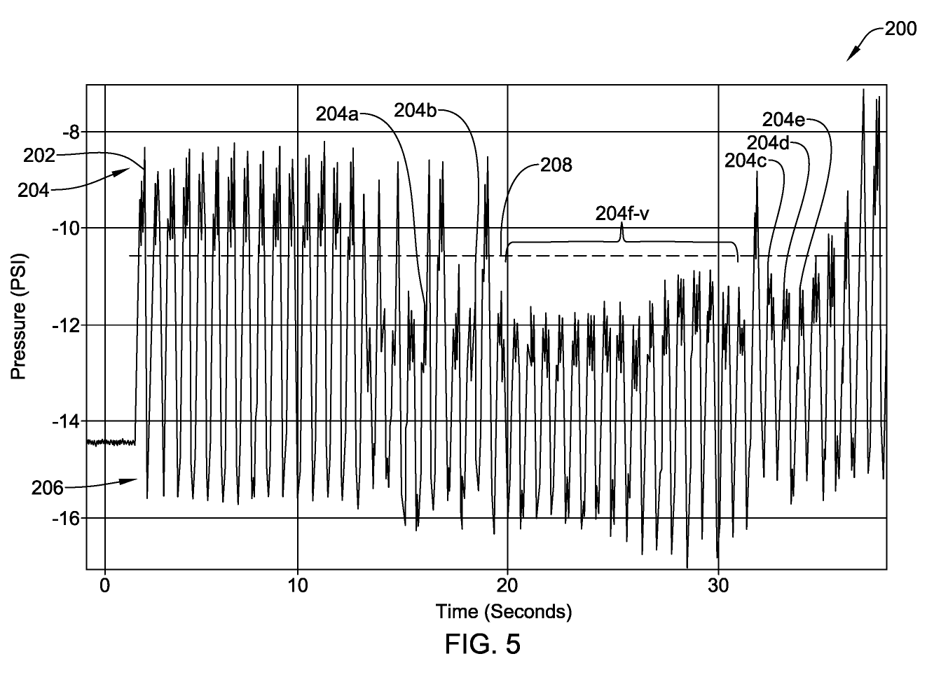

When the distal end of the effluent return tube 66 or catheter shaft is in contact with a clot or thrombus, the maximum pressure measured at the pressure sensor 162 may decrease as the suction required to move the effluent along the effluent return tube 66 is increased thus reducing a pressure. Additionally, the flow rate of effluent through the lumen 172 of the effluent return tube 66 may decrease contributing to the pressure decrease. As the thrombectomy system 10 is actively removing a clot or thrombus, increased negative pressure occurs again lowering the maximum pressure measured at the pressure sensor 162. During clot removal, the flow rate of effluent through the lumen 172 of the effluent return tube 66 may decrease contributing to the pressure decrease. The decrease in flow rate may be due to change in the viscosity of the effluent when removing a clot or thrombus, the ratio of blood to saline, or the like. FIG. 5 is an illustrative graph 200 of pressure data obtained at the pressure sensor 162 over time during a thrombectomy procedure. A pressure curve 202 may include a plurality of peaks or maximum pressures 204 and a plurality of valleys or minimum pressures 206. The peaks 204 and valleys 206 may occur due to the pulsatile nature of the roller pump 40 controlling the flow of effluent. The frequency of the pressure curve 202 may be directly proportional to a speed of the roller pump 40. When the pressure peak (or maximum pressure) 204 of an oscillation drops below or is less than a predetermined pressure threshold 208, the controller 33 of the thrombectomy system 10 may be configured to determine the distal end of the effluent return tube 66 or catheter shaft is in contact with a clot or thrombus. In some cases, a single peak 204a, 204b below or less than the predetermined pressure threshold 208 may be indicative of a clot or thrombus. Alternatively, or additionally, more than one consecutive peak 204c, 204d, 204e below or less than the predetermined pressure threshold 208 may be indicative of a clot or thrombus at or adjacent to the distal end of the effluent return tube 66 or catheter shaft. In some embodiments, when the maximum pressure (e.g., pressure peak 204) is below or less than the predetermined threshold pressure for a length of time greater than a first predetermined length of time but less than a second predetermined length of time, the controller 33 of the thrombectomy system 10 may be configured to determine the distal end of the effluent return tube 66 or catheter shaft is in contact with a clot or thrombus.

The controller 33 may be further configured to use the pressure data or pressure curve 202 to determine if the thrombectomy system 10 is actively removing a clot. In some cases, when a plurality of consecutive pressure peaks 204 (e.g., peaks 204f-v) are below or less than the predetermined pressure threshold 208, the controller 33 of the thrombectomy system 10 may be configured to determine the distal end of the effluent return tube 66 or catheter shaft is actively removing a clot or thrombus. In some cases, the controller 33 may be configured to use a predetermined number of consecutive pressure peaks 204 below or less than the predetermined pressure threshold 208 as a metric for determining whether or not active clot or thrombus removal is occurring. For example, two or more consecutive peaks 204, three or more consecutive peaks 204, four or more consecutive peaks 204, etc. may be indicative of active clot or thrombus removal. It is contemplated that the number of consecutive peaks used as the metric for determining active clot or thrombus removal is occurring may be greater than a number of consecutive peaks used as a metric for determining the distal end of the effluent return tube 66 or catheter shaft is in contact or with a clot or thrombus. In some embodiments, when the maximum pressure (e.g., pressure peak 204) is below or less than the predetermined threshold pressure for a length of time greater than the second predetermined length of time, the controller 33 of the thrombectomy system 10 may be configured to determine the thrombectomy system 10 is actively removing a clot or thrombus. The second predetermined length of time may be greater than the first predetermined length of time. For example, the maximum pressure 204 may remain under the predetermined pressure threshold 208 for a longer period of time when the thrombectomy system 10 is actively removing a clot than when the distal end of the effluent return tube 66 or catheter shaft is in contact with a clot or thrombus. In some embodiments, the controller 33 (or DAQ) may use additional or alternative features of the graph 200 to determine when the effluent return tube 66 is in contact with a clot or thrombus or when active clot or thrombus removal is occurring. For example, the controller 33 (or DAQ 166) may use Fourier transform to use the frequency of the pressure wave 202 to determine when the effluent return tube 66 is in contact with a clot or thrombus or when active clot or thrombus removal is occurring. For example, Fourier transform may show the shift in amplitude of the pressure peaks 204, from high amplitude (e.g. the distal end of the effluent return tube 66 is not in contact with a clot) to low amplitude (e.g. the distal end of the effluent return tube 66 is in contact with a clot). There may also be a frequency shift of the pressure peaks 204 that could be detected. Fourier series and Fast Fourier Transform (FFT) may also be used for signal processing. It is further contemplated that high- and/or low-pass filters could also be applied to the signal. This could reduce the noise observed in pressure readings, such as in FIG. 5. It is contemplated that using a high-pass and/or low-pass filter may create a smoother curve of the pressure signal that would be easier to identify when the distal end of the effluent return tube 66 is in contact with a clot and/or when the thrombectomy system 10 is actively removing a clot or thrombus. These are just some examples.

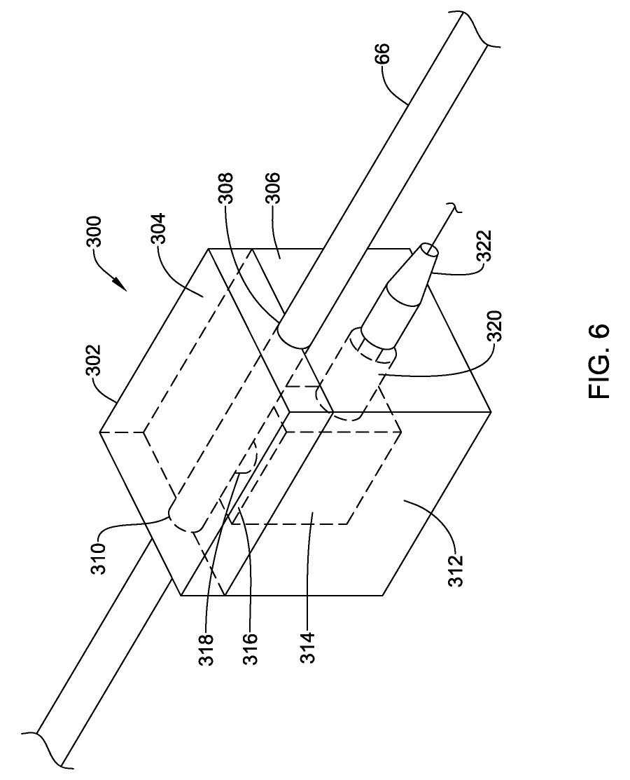

Alternatively, or additionally, a non-contact flow sensor may be used to monitor a flow of fluid through the lumen 172 of the effluent return tube 66 to determine when active clot or thrombus removal is occurring. For example, a non-contact flow sensor may not contact the fluid measured by the non-contact flow sensor. FIG. 6 is a perspective view of an illustrative non-contact flow sensor 300 positioned around an outer surface of the effluent return tube 66. It is contemplated that the non-contact flow sensor 300 may be positioned anywhere along the length of the effluent flow tube 66 that is exterior to the patient’s body. The non-contact flow sensor 300 may include a housing 302 having a first portion 304 and a second portion 306 releasably coupled to the first portion 304. The first and second portions 304, 306 may be releasably secured using snap fits, latches, friction fits, or the like to releasably secure the non-contact flow sensor 300 to the effluent return tube 66. In some cases, the first and second portions 304, 306 may be hingedly or pivotably coupled to one another. However, this is not required. When coupled, the first and second portions 304, 306 may define a first opening 308 and a second opening 310 for receiving the effluent return tube 66 through the first opening, through a cavity 312 defined by the first and second portions 304, 306, and through the second opening 310. In some cases, the openings 308, 310 may be configured to conform to an outer surface of the effluent return tube 66. However, this is not required.

The non-contact flow sensor 300 may include a load cell 314 disposed within the cavity thereof. The load cell 314 may include a loading platform 316 configured to receive the load or force. In some cases, an extension 318 may extend from the loading platform 316, although this is not required. The load cell 314 may be positioned within the housing 302 such that the outer surface of the effluent return tube 66 is in contact with the loading platform or the extension 318 if so provided. The effluent return tube 66 may expand and contract as the flow of fluid through the lumen 172 thereof changes. For example, an increasing volume or flow rate of fluid through the lumen 172 may expand the effluent return tube 66 and thus increase the force exerted on the loading platform 316. The load cell 314 may include a transducer that creates an electrical signal the magnitude of which may be proportional to the force exerted on the loading platform 316. The electrical signal may be transmitted to a data acquisition device (similar to DAQ 166) or to the controller 33 via an electrical connection 322. The electrical connection 322 may be releasably secured to an electrical port 320 of the load cell 314. When a separate DAQ is provided, the DAQ may be in communication with the controller 33 via a wired or wireless connection, as desired.

During clot removal, the flow rate of effluent through the lumen 172 of the effluent return tube 66 may decrease. The decrease in flow rate may be due to change in the viscosity of the effluent when removing a clot or thrombus, the ratio of blood to saline, or the like. The decrease in flow rate may be detected as a reduction in the force exerted on the loading platform 316. The controller 33 may be configured to detect the active removal of a clot or thrombus when the flow rate through the lumen 172 of the effluent return tube 66 falls below a predetermined threshold flow rate. In some cases, a reduction in the flow rate through the lumen of the effluent return tube 66 may also occur when the distal tip of the effluent return tube 66 or catheter shaft comes into contact with a clot or thrombus. It is contemplated that the controller 33 may be configured to distinguish between contact with a clot or thrombus and active removal of a clot or thrombus by a length of time the flow rate is less than or below the predetermined threshold flow rate. For example, when the flow rate is less than or below the predetermined threshold flow rate for a length of time greater than a first predetermined length of time but less than a second predetermined length of time, the controller 33 of the thrombectomy system 10 may be configured to determine the distal end of the effluent return tube 66 or catheter shaft is in contact with a clot or thrombus. When the flow rate is less than or below the predetermined threshold flow rate for a length of time greater than the second predetermined length of time, the controller 33 of the thrombectomy system 10 may be configured to determine that active clot or thrombus removal is occurring.

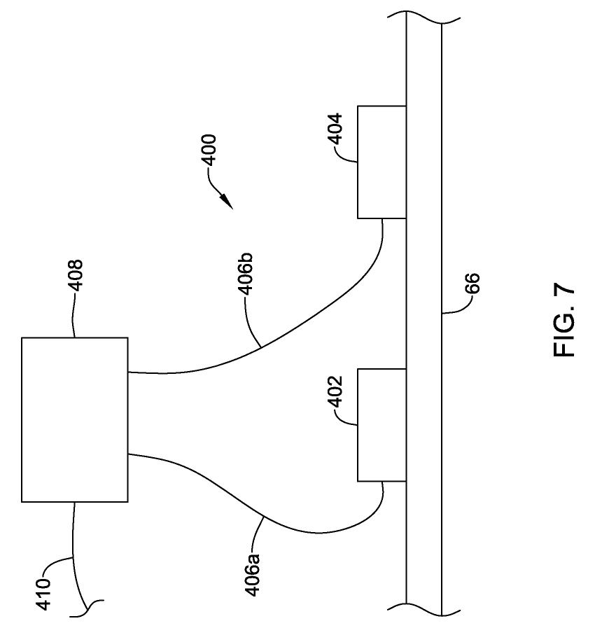

FIG. 7 is a perspective view of another illustrative non-contact flow sensor 400 that may be used to determine a flow rate of effluent through the lumen 172 of the effluent return tube 66. In some embodiments, the non-contact flow sensor 400 may be an ultrasound doppler flow meter. The non-contact flow sensor 400 may be configured to emit ultrasound waves into the fluid (e.g., effluent) flowing through the effluent return tube 66 which are then reflected. The frequency shift between the transmitted and reflected waves (caused by the movement of the fluid) is used to calculate the flow rate. It is contemplated that the effluent return tube 66 may be made from a clear or transparent plastic such that the emission of sound through the wall of the effluent return tube 66 is feasible. Generally, the non-contact flow sensor 400 may be releasably secured to or clamped on to an outer surface of the effluent return tube 66. It is contemplated that the non-contact flow sensor 400 may be positioned anywhere along the length of the effluent flow tube 66 that is exterior to the patient’s body. The non-contact flow sensor 400 may include at least one transmitter 402 and at least one receiver 404. The longitudinal and/or circumferential spacing of the transmitter 402 and the receiver 404 may vary depending on the type and/or configuration of the ultrasound doppler flow meter. In some cases, the non-contact flow sensor 400 may include more than one transmitter 402 and/or more than one receiver 404. Each of the transmitter 402 and the receiver 404 may be electrically coupled to a data acquisition device (DAQ) 408 through one or more electrical connections 406a, 406b. However, in some cases, the transmitter 402 and the receiver 404 may be directly coupled to the controller 33. When a separate DAQ 408 is provided, the DAQ 408 may be in communication with the controller 33 via a wired connection 410 or wireless connection, as desired.

As described above, during clot removal, the flow rate of effluent through the lumen 172 of the effluent return tube 66 may decrease. The decrease in flow rate may be due to change in the viscosity of the effluent when removing a clot or thrombus, the ratio of blood to saline, or the like. The decrease in flow rate may be detected as a frequency shift in the transmitted and reflected waves at the non-contact flow sensor 400. The controller 33 may be configured to detect the active removal of a clot or thrombus when the flow rate through the lumen 172 of the effluent return tube 66 falls below a predetermined threshold flow rate. In some cases, a reduction the flow rate through the lumen of the effluent return tube 66 may also occur when the distal tip of the effluent return tube 66 or catheter shaft comes into contact with a clot or thrombus. It is contemplated that the controller 33 may be configured to distinguish between contact with a clot or thrombus and active removal of a clot or thrombus by a length of time the flow rate is less than or below the predetermined threshold flow rate. For example, when the flow rate is less than or below the predetermined threshold flow rate for a length of time greater than a first predetermined length of time but less than a second predetermined length of time, the controller 33 of the thrombectomy system 10 may be configured to determine the distal end of the effluent return tube 66 or catheter shaft is in contact with a clot or thrombus. When the flow rate is less than or below the predetermined threshold flow rate for a length of time greater than the second predetermined length of time, the controller 33 of the thrombectomy system 10 may be configured to determine that active clot or thrombus removal is occurring.

In some cases, the detection of a clot or thrombus or the active removal of a clot or thrombus may be used to automatically control operation of the thrombectomy system 10. For example, when the data from the pressure sensor 162 and/or the non-contact flow sensors 300, 400 are received at the controller 33, the controller 33 may adjust the control parameters of the system 10 in response to the presence or lack thereof of a clot or thrombus. For example, based on the data, aspiration may be started or stopped, a cycle rate of the inflow pump 56 may be increased or decreased, a frequency of the roller pump 40 may be increased or decreased, the roller pump 40 may be incrementally opened or closed, or the like. Alternatively, or additionally, the detection of a clot or thrombus or the active removal of a clot or thrombus may be used to display alerts and/or notifications to the user at the user interface 32. Additionally, or alternatively, the user may be alerted to the detection of a clot or thrombus or the active removal of a clot or thrombus through the use of visual alerts or lights (e.g., on/off, blinking, differing colors, or the like), audible alerts or sounds (e.g., beeps, buzzers, spoken words, or the like), haptics, or the like which may not require to the user to view the user interface 32. The user may then manually alter control parameters based on the information displayed.

In some embodiments, the pressure sensor 162 and the non-contact flow sensors 300, 400 may be provided as standalone systems. For example, each of the pressure sensor 162 and the non-contact flow sensors 300, 400 may have a DAQ (e.g., DAQ 166, 408) which processes the electrical signal from said device and displays or otherwise alerts the user of the pressure or flow rate for the user. The user may then use the displayed pressure or flow rate to determine if the distal end of the effluent return tube 66 is in contact with a clot or thrombus or if active clot or thrombus removal is occurring. Alternatively, or additionally, the DAQ 166, 408 of the pressure sensor 162 or non-contact flow sensors 300, 400 may be configured to determine and display when the distal end of the effluent return tube 66 is in contact with a clot or thrombus or if active clot or thrombus removal is occurring.

In some configurations, the thrombectomy system 10 may include two or more different systems for determining the presence of a clot or thrombus or when active removal is occurring. For example, the system 10 may include a pressure sensor 162 coupled to the transition fixture and a non-contact flow sensor 300, 400 coupled to the effluent return tube 66. The DAQ e.g., DAQ 166, 408 or the controller 33 may be configured to use the data from two or more sensors to determine the presence of a clot or thrombus or when active removal is occurring. It is contemplated that the use of two or more sensors may increase the sensitivity of the readings.

The materials that can be used for the various components of the thrombectomy catheter, pump/catheter assembly, and/or other devices disclosed herein may include those commonly associated with medical devices.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A thrombectomy catheter, comprising:
an effluent return tube comprising a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube configured for communication with a fluid source near the catheter body proximal end region;
a transition fixture coupled to the proximal end region of the catheter body, the transition fixture including at least one port; and
a sensor coupled relative to the catheter body and configured to monitor a flow of fluid through the catheter lumen.

2. The thrombectomy catheter of claim 1, wherein the sensor is a pressure sensor.

3. The thrombectomy catheter of claim 2, wherein the pressure sensor is secured to the at least one port.

4. The aspiration catheter of claim 2, wherein the pressure sensor is releasably coupled to the at least one port.

5. The thrombectomy catheter of claim 2, wherein the pressure sensor is configured to measure a pressure of a fluid in the catheter lumen.

6. The thrombectomy catheter of claim 1, wherein the sensor is a non-contact flow sensor.

7. The thrombectomy catheter of claim 6, wherein the non-contact flow sensor is in contact with an outer surface of the catheter body.

8. The thrombectomy catheter of claim 7, wherein the non-contact flow sensor is releasably secured to the outer surface of the catheter body.

9. The thrombectomy catheter of claim 6, wherein the non-contact flow sensor comprises a load cell.

10. The thrombectomy catheter of claim 9, wherein the load cell is disposed within a housing.

11. The thrombectomy catheter of claim 9, wherein a loading platform of the load cell is in contact with the outer surface of the catheter body.

12. The thrombectomy catheter of claim 6, wherein the non-contact flow sensor comprises an ultrasound doppler flow meter.

13. The thrombectomy catheter of claim 6, wherein the non-contact flow sensor is configured to measure a flow rate of a fluid in the catheter lumen.

14. The thrombectomy catheter of claim 1, further comprising a data acquisition device electronically coupled to the sensor.

15. A thrombectomy system, comprising:
a processor coupled to a pump and a thrombectomy catheter, wherein the thrombectomy catheter includes:
an effluent return tube comprising a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube configured for communication with a fluid source near the catheter body proximal end region;
a sensor releasably coupled relative to the catheter body and configured to monitor a flow of fluid through the catheter lumen.

16. The thrombectomy system of claim 15, wherein the processor is operably coupled to the sensor.

17. A method for detecting a thrombus, the method comprising:
inserting a thrombectomy catheter into a lumen, the thrombectomy catheter comprising:
an effluent return tube comprising a catheter body extending from a proximal end region to a distal end region and including a catheter lumen extending between the proximal end region and the distal end region;
a high-pressure fluid supply tube extending through the catheter lumen from the catheter body proximal end region toward the catheter body distal end region, the high-pressure fluid supply tube configured for communication with a fluid source near the catheter body proximal end region; and
a sensor releasably coupled relative to the catheter body and configured to monitor a flow of fluid through the catheter lumen;
determining a pressure or a flow rate of the fluid in the catheter lumen from a signal from the sensor;
determining the distal end region of the catheter body is in contact with a thrombus when the pressure or flow rate is less than a predetermined threshold for a first predetermined length of time.

18. The method of claim 17, further comprising determining the thrombectomy catheter is actively removing the thrombus when the pressure or flow rate is less than the predetermined threshold for a second predetermined length of time greater than the first predetermined length of time.

19. The method of claim 17, wherein the sensor is a pressure sensor.

20. The method of claim 17, wherein the sensor is a non-contract flow sensor.
